(19) United States
(12) Patent Application Publication
(10) Pub. No.: US 2023/0134045 A1
(43) Pub. Date: May 4, 2023

(54) METHOD AND KIT FOR EVALUATING PROGNOSIS IN PATIENT WITH BREAST CANCER

(71) Applicants: National Tsing Hua University, Hsinchu (TW); Taipei Medical University, Taipei City (TW); CHANG GUNG MEMORIAL HOSPITAL, LINKOU, Taoyuan City (TW)

(72) Inventors: Wen-Ching Wang, Hsinchu (TW); Che-Chang Chang, Taipei city (TW); Wen-Ling Kuo, Taoyuan City (TW)

(21) Appl. No.: 17/728,224

(22) Filed: Apr. 25, 2022

(30) Foreign Application Priority Data
Oct. 26, 2021 (TW) 110139746

(51) Int. Cl. G01N 33/574; C12Q 1/6886

(52) U.S. Cl. CPC G01N 33/57415; C12Q 1/6886; C12Q 2600/118; C12Q 2600/158; G01N 2800/52

(57) ABSTRACT

The present disclosure provides a method for evaluating prognosis in a patient with breast cancer, including the following steps: (a) detecting the expression level of pyruvate kinase in a sample from the patient; (b) detecting the expression level of O-linked β-N-acetylglucosamine in the sample; and (c) evaluating a clinical treatment score post-5 years, wherein high expression levels of both the pyruvate kinase and the O-GlcNAc, and high score of the CTS5 indicate that the patient has a poor prognosis. The present disclosure also provides a kit for evaluating prognosis in a patient with breast cancer.

### CROSS-REFERENCE TO RELATED APPLICATION

This application claims priority of Taiwan patent application No. 110139746, filed on Oct. 26, 2021, the content of which is incorporated herein in its entirety by reference.

### BACKGROUND OF THE INVENTION

### 1. Field of the Invention

The present invention relates to a method and a kit for evaluating prognosis in a patient with breast cancer.

### 2. The Prior Art

Breast cancer is a highly heterogeneous tumor and can be classified clinically based on its molecular characteristics. Different types of breast cancer have different tumor progression, treatment methods, recurrence and prognosis. How to effectively determine the risk of breast cancer recurrence and prognosis to select the appropriate treatment is an important clinical topic.

The early Nottingham prognostic index (NPI) and the later developed clinical treatment score post-5 years (CTS5) are all based on clinical characteristics as the criteria for determining prognosis. However, studies have reported that the prediction accuracy of CTS5 is lower in patients aged younger than 50 years.

Several genetic examining methods are used to evaluate prognosis in a patient with breast cancer and the sensitivity to chemotherapy, such as OncotypeDx, MammaPrint, EndoPredict, Prosigna, and breast cancer index (BCI). However, these methods are all based on the analysis of ribonucleic acid (RNA) expressions for a specific gene, and may not truly reflect the actual expressions of protein markers. It is impossible to provide clear mechanical instructions for the treatment of drugs.

In order to solve the above-mentioned problems, those skilled in the art urgently need to develop a novel method and kit for evaluating prognosis in a patient with breast cancer for the benefit of a large group of people in need thereof.

### SUMMARY OF THE INVENTION

A primary objective of the present invention is to provide a method for evaluating prognosis in a patient with breast cancer, comprising the following steps: (a) detecting an expression level of pyruvate kinase in a sample from the patient; (b) detecting an expression level of O-linked β-N-acetylglucosamine (O-GlcNAc) in the sample of step (a); and (c) evaluating a clinical treatment score post-5 years (CTS5); wherein high expression levels of both the pyruvate kinase and the O-GlcNAc, and high score of the CTS5 indicate that the patient has a poor prognosis.

According to an embodiment of the present invention, the pyruvate kinase is pyruvate kinase isoenzyme M2 (PKM2).

According to an embodiment of the present invention, the breast cancer is a luminal breast cancer that is hormone receptor (HR) positive and human epidermal growth factor receptor type 2 (HER2) negative.

According to an embodiment of the present invention, the HR positive is selected from the group consisting of: estrogen receptor (ER) positive, progesterone receptor (PR) positive, and a combination thereof.

According to an embodiment of the present invention, the expression level of pyruvate kinase in step (a) and the expression level of O-GlcNAc in step (b) are detected by an immunohistochemistry (IHC) method.

According to an embodiment of the present invention, the prognosis is evaluated by disease-free survival (DFS).

According to an embodiment of the present invention, the DFS is ten years.

According to an embodiment of the present invention, the expression level of PKM2 and the expression level of O-GlcNAc are determined as high expression levels or low expression levels by analysis of Tumor Immune Estimation Resource (TIMER).

According to an embodiment of the present invention, the expression level of PKM2 and the expression level of O-GlcNAc are determined as high expression levels based on top 25% of patients with breast cancer as criterion.

Another objective of the present invention is to provide a kit for evaluating prognosis in a patient with breast cancer, comprising: a first test reagent for detecting an expression level of pyruvate kinase in a sample from the patient; a second test reagent for detecting an expression level of O-linked β-N-acetylglucosamine (O-GlcNAc), wherein the second test reagent interacts with the first test reagent; and a third test reagent for evaluating a clinical treatment score post-5 years (CTS5), wherein the third test reagent interacts with the first test reagent and the second test reagent; wherein high expression levels of both the pyruvate kinase and the O-GlcNAc, and high score of the CTS5 indicate that the patient has a poor prognosis.

According to an embodiment of the present invention, the pyruvate kinase is pyruvate kinase isoenzyme M2 (PKM2).

According to an embodiment of the present invention, the breast cancer is a luminal breast cancer that is hormone receptor (HR) positive and human epidermal growth factor receptor type 2 (HER2) negative.

According to an embodiment of the present invention, the HR positive is selected from the group consisting of: estrogen receptor (ER) positive, progesterone receptor (PR) positive, and a combination thereof.

In summary, the method and the kit of the present invention have the effect on providing a new combination of clinical molecular characteristics for more effective evalution of the prognosis in a patient with breast cancer, especially for the luminal breast cancer that is hormone receptor (HR) positive and human epidermal growth factor receptor type 2 (HER2) negative. In addition, the method of the present invention is more effective than using CTS5 alone, and can become a new metabolic prognostic biomarker, as well as a concomitant indicator of drug use.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1A is a survival curve diagram showing that high expression of pyruvate kinase isoenzyme M2 (PKM2) is significantly associated with poor clinical prognosis in luminal breast cancer, in which OS represents 10-year overall survival.

FIG. 1B is a survival curve diagram showing that high expression of PKM2 is significantly associated with poor clinical prognosis in luminal breast cancer, in which RFS represents relapse-free survival.

FIG. 2A is a survival curve diagram showing survival analysis for hormone receptor (HR)⁺ luminal breast cancer at Chang Gung Memorial Hospital (2005-2013), in which Lum A breast cancer represents the luminal breast cancer that is hormone receptor (HR) positive (estrogen receptor (ER) and/or progesterone receptor (PR)⁺, Ki67≤ 20%, or grade 1) and human epidermal growth factor receptor type 2 (HER2) negative; Lum B breast cancer represents the luminal breast cancer that is HR positive (ER and/or PR⁺, Ki67 > 20%, or grade 2-3) and HER2 negative.

FIG. 2B is a survival curve diagram showing disease-free survival (DFS) analysis for HR⁺ luminal breast cancer at Chang Gung Memorial Hospital (2005-2013), in which Lum A breast cancer represents the luminal breast cancer that is HR positive (ER and/orPR⁺, Ki67≤ 20%, or grade 1) and HER2 negative; Lum B breast cancer represents the luminal breast cancer that is HR positive (ER and/or PR⁺, Ki67 > 20%, or grade 2-3) and HER2 negative.

FIG. 3A is a representative image of immunohistochemistry (IHC) profiles, reviewed blindly by two pathologists (CJ Chen and YC Hsu) based on two parameters: the staining intensity score (0 = negative; 1 = weak; 2 = moderate; and 3 = strong), and the percentage of immunopositive cells (0-100); the Q score (0-300) was calculated by multiplying these two parameters; two groups were stratified based on the Q score: low expression (Q□ mean) and high expression (Q > mean); Kaplan-Meier analysis was conducted to evaluate the clinical relevance; Low represents Q≤ mean; High represents Q > mean; the framed region was magnified, and the magnified images are shown in the lower right corner.

FIG. 3B is a data diagram showing that high O-GlcNAc expression is significantly associated with the risk of recurrence; IHC results were reviewed blindly by two pathologists (CJ Chen and YC Hsu) based on two parameters: the staining intensity score (0 = negative; 1 = weak; 2 = moderate; and 3 = strong), and the percentage of immunopositive cells (0-100); the Q score (0-300) was calculated by multiplying these two parameters; two groups were stratified based on the Q score: low expression (Q≤ mean) and high expression (Q > mean); Kaplan-Meier analysis was conducted to evaluate the clinical relevance; Low represents Q≤ mean; High represents Q > mean.

FIG. 3C is a data diagram showing that high PKM2 expression is significantly associated with the risk of recurrence; IHC results were reviewed blindly by two pathologists (CJ Chen and YC Hsu) based on two parameters: the staining intensity score (0 = negative; 1 = weak; 2 = moderate; and 3 = strong), and the percentage of immunopositive cells (0-100); the Q score (0-300) was calculated by multiplying these two parameters; two groups were stratified based on the Q score: low expression (Q≤ mean) and high expression (Q > mean); Kaplan-Meier analysis was conducted to evaluate the clinical relevance; Low represents Q≤ mean; High represents Q > mean.

FIG. 3D is an overall survival curve diagram showing the expression level according to O-GlcNAc, and overall survival of patients with ER⁺/HER2⁻ luminal breast cancer, in which OS represents overall survival.

FIG. 3E is a survival curve diagram showing the expression level according to PKM2, and overall survival of patients with ER⁺/HER2⁻ luminal breast cancer, in which OS represents overall survival.

FIG. 3F is a survival curve diagram showing the expression levels according to O-GlcNAc and PKM2, and overall survival of patients with ER⁺/HER2⁻ luminal breast cancer, in which OS represents overall survival.

FIG. 3G is a disease-free survival (DFS) curve diagram showing that high expression of O-GlcNAc can predict poor prognosis in HR⁺/HER2⁻ breast cancer, in which DFS represents disease-free survival.

FIG. 3H is a survival curve diagram showing that high expression of PKM2 can predict poor prognosis in HR⁺/HER2⁻ breast cancer, in which DFS represents disease-free survival.

FIG. 3I is a survival curve diagram showing that high expressions of PKM2 and O-GlcNAc can predict poor prognosis in HR⁺/HER2⁻ breast cancer, in which DFS represents disease-free survival.

FIG. 4 is a data diagram showing logistic regression and receiver operating characteristic (ROC) curve analysis in HR⁺/HER2- luminal breast cancer, in which values in parentheses refer to the area under curve (AUC); PKM2 represents pyruvate kinase isoenzyme M2; O-GlcNAc represents O-linked β-N-acetylglucosamine; CTS5 represents clinical treatment score post-5 years; PKM2+O-GlcNAc represents PKM2 in combination with O-GlcNAc; PKM2+O-GlcNAc+CTS5 represents PKM2 in combination with O-GlcNAc and CTS5.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

According to the present invention, the expression level of PKM2 and the expression level of the O-GlcNAc are determined as high expression levels based on top 25% of subjects with breast cancer as criterion.

According to the present invention, clinical treatment score post-5 years (CTS5) is a calculation tool integrating four clinical data including tumor size, tumor grade, patient age, and the number of nodes to estimate the risk of breast cancer.

According to the present invention, Chi-square test was performed to compare clinicopathological properties of the patients with categorical variables. Kaplan-Meier analysis was conducted to assess overall patient survival, which was estimated with the time from diagnosis until death or until last follow-up. The log-rank test was used to determine the statistical significance between groups. Differences were considered significant at p < 0.05. All p values were obtained from 2-sided tests. Logistic regression analysis was used to assess the contribution of the expression of O-GlcNAc and PKM2, and/or the CTS5 score in prognosis. Receiver operating characteristic (ROC) curve was plotted based on the set of sensitivity and specificity. Area under the curve (AUC) was computed using numerical integration of the ROC curves. Statistical analysis was performed using IBM SPSS, version 25 (IBM Corp.).

### Example 1

### High Expression of Pyruvate Kinase Isoenzyme M2 (PKM2) is Associated With Poor Clinical Outcome in Luminal Breast Cancer

The protocols in this example and the following examples have been approved by the Institutional Review Board of Chang-Gung Memorial Hospital (CGMH) (IRB#201700716A3), and all experiments were performed in accordance with the approved guidelines. Written informed consent was obtained from each patient.

A total of 3166 hormone receptor (HR)⁺/ human epidermal growth factor receptor type 2 (HER2)⁻ luminal breast cancer patients who had received therapy at Chang-Gung Memorial Hospital (CGMH), Linkou, Taiwan (2005-2013) were reviewed based on the pathological and clinical properties. The diagnostic criteria for HR⁺/HER2⁻ luminal breast cancer was based on the receptor status (estrogen receptor (ER), progesterone receptor (PR), and HER2) by immunohistochemistry (IHC) and HER2 amplification by fluorescence in situ hybridization (FISH) in invasive cancer as the following: HR⁺, more than 1% positive for ER or PR; HER2⁻, an IHC score of 0 to 1⁺, or 2⁺ with non-amplified HER2 by FISH. One hundred sixty-nine HR⁺/HER2⁻ patients who had concluding pathologic reports and no other malignancies were firstly selected (retrospective cohort study from 2001 to 2017). Seven patients were excluded for the following reasons: 4 cases with incomplete initial diagnostic information, 2 with mucinous carcinoma, and 1 with no available formalin-fixed paraffin embedded block of the primary tumor. The final number of enrollments was 162, including 50 patients with documented recurrence, and 112 patients without any recurrence over 10 years. Clinical properties of patients are shown in Table 1. Demographic factors of this retrospective cohort included age, history of diabetes, ER and PR status, tumor size, grade, TN stage, surgical treatment, chemotherapy use, endocrine therapy and radiotherapy. The time and location of recurrence were documented.

TABLE 1

| Factors | | Non-recurrent No. (%), n = 112 | Recurrent No. (%), n = 50 | p value |
|---|---|---|---|---|
| Age (years) | Median (IQR) | 51.0 (16.0) | 53.0 (22.0) | 0.517 |
| Age (years) | ≤ 50 | 51 (45.5) | 20 (40.0) | 0.512 |
| | > 50 | 61 (54.5) | 30 (60.0) | |
| Diabetes | No | 104 (92.9) | 45 (90.0) | 0.542 |
| mellitus | Yes | 8 (7.1) | 5 (10.0) | |
| Operation type | Mastectomy | 62 (55.4) | 27 (54.0) | 0.873 |
| | Breast | 50 (44.6) | 23 (46.0) | |
| | conservation | | | |
| Invasive tumor | Median (IQR) | 1.9 (1.4) | 2.4 (1.7) | 0.019 |
| size (cm) | | | | |
| SBR grade | 1 | 13 (11.6) | 5 (10.0) | 0.806 |
| | 2 | 61 (54.5) | 30 (60.0 ) | |
| | 3 | 38 (33.9) | 15 (30.0) | |
| Estrogen | Negative | 4 (3.6) | 0 | 0.312 |
| receptor | Positive | 108 (96.4) | 50 (100.0) | |
| Progesterone | Negative | 11 (9.8) | 3 (6.0) | 0.553 |
| receptor | Positive | 101 (90.2) | 47 (94.0) | |
| T stage | T1a | 0 | 1 (2.0) | 0.014 |
| | T1b | 8 (7.1) | 1 (2.0) | |
| | T1c | 54 (48.2) | 14 (28.0) | |
| | T2 | 45 (40.2) | 30 (60.0) | |
| | T3 | 5 (4.5) | 3 (6.0) | |
| | T4 | 0 | 1 (2.0) | |
| N stage | N0 | 56 (50.0) | 21 (42.0) | 0.353 |
| | N1 | 40 (35.7) | 22 (44.0) | |
| | N2 | 12 (10.7) | 3 (6.0) | |
| | N3 | 4 (3.6) | 4 (8.0) | |
| Stage | I | 40 (35.7) | 9 (18.0) | 0.067 |
| | II | 55 (49.1) | 33 (66.0) | |
| | III | 17 (15.2) | 8 (16.0) | |
| Chemotherapy | No | 19 (17.0) | 7 (14.0) | 0.635 |
| | Yes | 93 (83.0) | 43 (86.0) | |
| Hormone | No | 3 (2.7) | 2 (4.0) | 0.645 |
| therapy | Yes | 109 (97.3) | 48 (96.0) | |
| Radiotherapy | No | 57 (50.9) | 23 (46.0) | 0.565 |
| | Yes | 55 (49.1) | 27 (54.0) | |

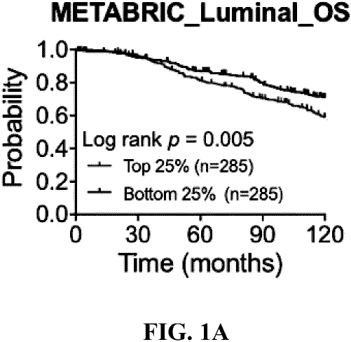

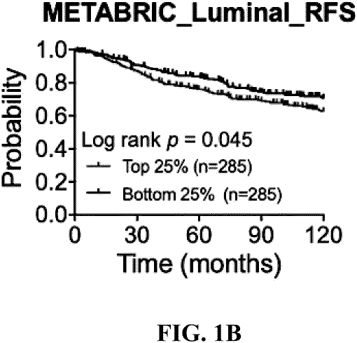

PKM2 is overexpressed in the breast tumors and associated with poor breast cancer prognosis in a meta-analysis. We sought to further evaluate the clinical relevance of PKM2 expression in the luminal tumors using the Metabric cohort defined by PAM50. Data were obtained from METABRIC online dataset. The mRNA expression levels of PKM2 were divided in quartiles. An analysis between the top quartile group (top 25%) and the bottom one (bottom 25%) was then compared by log-rank test. FIG. 1A shows that patients with high PKM2 expression exhibited a significantly worse 10-year survival than did those with low PKM2 expression (top 25% vs. bottom 25%, p = 0.005). A significantly worse relapse-free survival was also found for the top-quartile group as compared with the bottom-quartile one (top 25% vs. bottom 25%, p = 0.045). These results suggested high expression of PKM2 is associated with worse clinical outcome in the luminal tumors.

### Example 2

### High Expression of O-Linked β-N-Acetylglucosamine (O-GlcNAc) and PKM2 Can Predict Poor Prognosis of HR⁺/HER2⁻ Breast Cancer

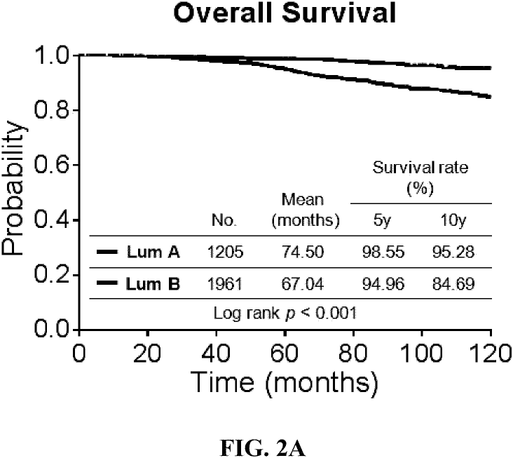

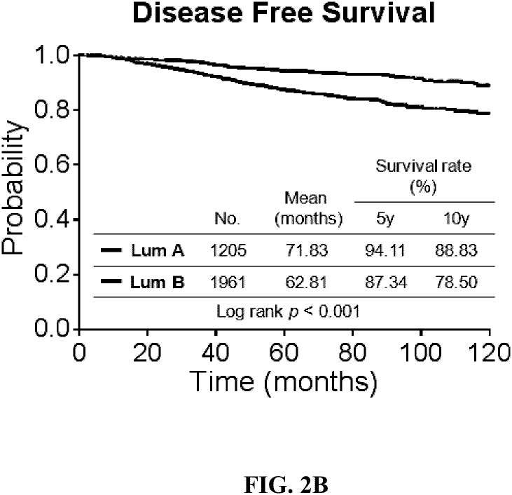

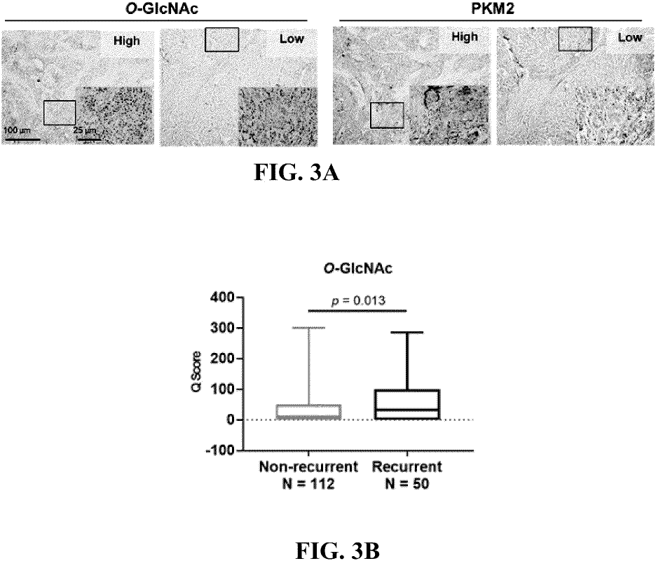

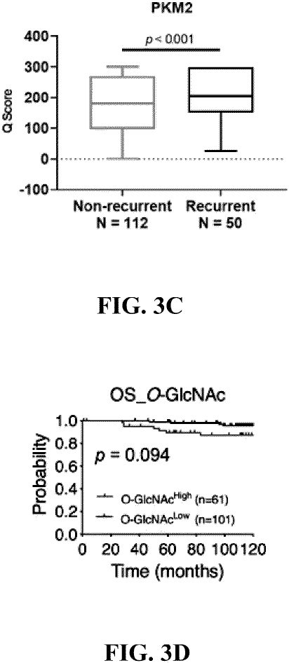

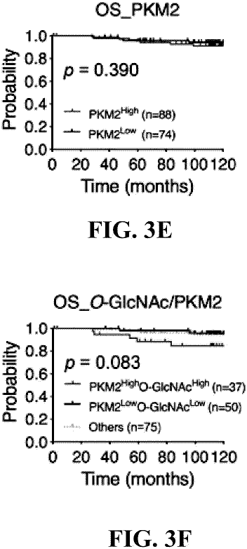

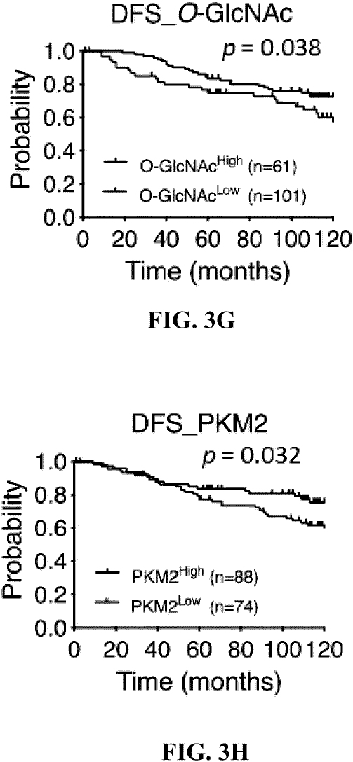

The procedure of immunohistochemistry (IHC) assessment used in this example is as follows. Consecutive paraffin-embedded serial sections of specimens (n = 162) were obtained from CGMH for single IHC staining of PKM2 and O-GlcNAc. The specimens were deparaffinized and rehydrated with xylene (Sigma-Aldrich) and ethanol (100% ethanol for 10 min, 95% ethanol for 5 min, 80% ethanol for 3 min, 70% ethanol for 3 min, 50% ethanol for 2 min, and then deionized water for 5 min). For antigen retrieval, specimens were incubated with citrate buffer at pH 6.0 (10 mM citrate, 0.05% Tween 20) and autoclaved at 121° C. for 20 min. The immunostaining procedures were performed using Novolink Polymer Detection System (Leica) according to the manufacturer’s manual. In brief, tissues were stained with anti-O-GlcNAc or anti-PKM2 for 60 min at room temperature. The working dilution of primary antibody was 1:50 for anti-O-GlcNAc (838004, Biolegend, San Diego, CA, USA) and 1:100 for anti-PKM2 (D78A4, Cell Signaling Technology, Danvers, MA, USA). IHC results were reviewed blindly by two pathologists (CJ Chen and YC Hsu) based on two parameters: the staining intensity score (0 = negative; 1 = weak; 2 = moderate; and 3 = strong), and the percentage of immunopositive cells (0-100). The Q score (0-300) was calculated by multiplying these two parameters. Two groups were stratified based on the Q score: low expression (Q ≤ mean) and high expression (Q > mean). Kaplan-Meier analysis was conducted to evaluate the clinical relevance. The experimental results are shown in FIGS. 2A-3D.

Patients with HR⁺/HER2⁻ luminal breast cancer (n = 3166, 2005-2013 at Chang Gung Memorial Hospital) were classified into two groups based on pathological and IHC features: luminal A (ER and/or PR⁺, Ki67 ≤ 20%, or grade 1) and luminal B (ER and/or PR⁺, Ki67 > 20%, or grade 2-3). The luminal B group exhibited a significantly lower survival rate than that of the luminal A group [10-year overall survival (OS), p < 0.001; 10-year DFS, p < 0.001] (FIGS. 2A and 2B), wherein p values (high vs. low) were determined by log-rank test, revealing the substantial outcome difference between the two subtypes in our hospital-based population.

We next evaluated the clinical relevance of O-GlcNAc and/or PKM2 expression in HR⁺/HER2⁻ luminal breast cancer. We enrolled 162 subjects (non-recurrent, n = 112, median [interquartile range (IQR)] age, 51.0 [16.0] years; recurrent, n = 50, median [IQR] age, 53.0 [22.0] years) who had received breast cancer therapy and were followed up until December, 2018 (median period, 123.4 months, IQR, 107.4-134.1 months) (Table 1). O-GlcNAc and PKM2 were characterized by IHC for each of the samples (FIG. 3A). FIG. 3A is a representative image of IHC profiles, reviewed blindly by two pathologists (CJ Chen and YC Hsu) based on two parameters: the staining intensity score (0 = negative; 1 = weak; 2 = moderate; and 3 = strong), and the percentage of immunopositive cells (0-100)(see Sung, W. W. et al. A polymorphic -844T/C in FasL promoter predicts survival and relapse in non-small cell lung cancer. Clin Cancer Res 17, 5991-5999, doi:10.1158/1078-0432.CCR-11-0227 (2011); Chen, C. J. et al. High expression of interleukin 10 might predict poor prognosis in early stage oral squamous cell carcinoma patients. Clin Chim Acta 415, 25-30, doi:10.1016/j.cca.2012.09.009 (2013)). The Q score (0-300) was calculated by multiplying these two parameters. Two groups were stratified based on the Q score: low expression (Q≤ mean) and high expression (Q > mean); Kaplan-Meier analysis was conducted to evaluate the clinical relevance; Low represents Q≤ mean; High represents Q > mean; the framed region was magnified, and the magnified images are shown in the lower right corner.

To assess the association of O-GlcNAc and PKM2 in recurrence, patients were divided into two groups, with or without recurrent status. A group comparison shows that high O-GlcNAc (p = 0.013) or high PKM2 (p < 0.001) is positively associated with risk of recurrence (FIGS. 3B and 3C ). FIG. 3B is a data diagram showing that high O-GlcNAc expression is significantly associated with the risk of recurrence, wherein p values were determined by two-tailed Student’s t test. FIG. 3C is a data diagram showing that high PKM2 expression is significantly associated with the risk of recurrence, wherein p values were determined by two-tailed Student’s t test.

Furthermore, samples were stratified into two groups using the mean Q score as a threshold. A Kaplan-Meier survival analysis revealed that high expression of O-GlcNAc (O-GlcNAc^{High}, p = 0.094), PKM2 (PKM2^{High}, p = 0.390), or the combined double-marker status (O-GlcNAc^{Hlgh}PKM2^{Hlgh} vs. O-GlcNAc^{Low}PKM2^{Low}, p = 0.083) were associated with worse overall survival, despite no statistical significance (FIGS. 3D-3F), wherein p values (high vs. low) were determined by log-rank test, and OS represents overall survival. Interestingly, high expression levels of O-GlcNAc (O-GlcNAc^{High}, p = 0.038) or PKM2 (PKM2^{High}, p = 0.032) were significantly associated with worse DFS (FIGS. 3G and 3H). FIG. 3G is a disease-free survival (DFS) curve diagram showing that high expression of O-GlcNAc can predict poor prognosis in HR⁺/HER2⁻ breast cancer, in which p values (high vs. low) were determined by log-rank test, and DFS represents disease-free survival. FIG. 3H is a survival curve diagram showing that high expression of PKM2 can predict poor prognosis in HR⁺/HER2⁻ breast cancer, in which, in which p values (high vs. low) were determined by log-rank test, and DFS represents disease-free survival. Importantly, the combined double-marker status offered an even greater statistical significance (O-GlcNAc^{High}PKM2^{High} vs. O-GlcNAc^{Low}PKM2^{Low}, p = 0.004) (FIG. 3I). FIG. 3I is a survival curve diagram showing that high expressions of PKM2 and O-GlcNAc can predict poor prognosis in HR⁺/HER2⁻ breast cancer, in which p values (high vs. low) were determined by log-rank test, and DFS represents disease-free survival.

These results of this example suggest that high expression of O-GlcNAc and PKM2 are associated with high risk of recurrence and worse DFS.

### Example 3

### Logistic Regression and Receiver Operating Characteristic (ROC) Curve Analysis

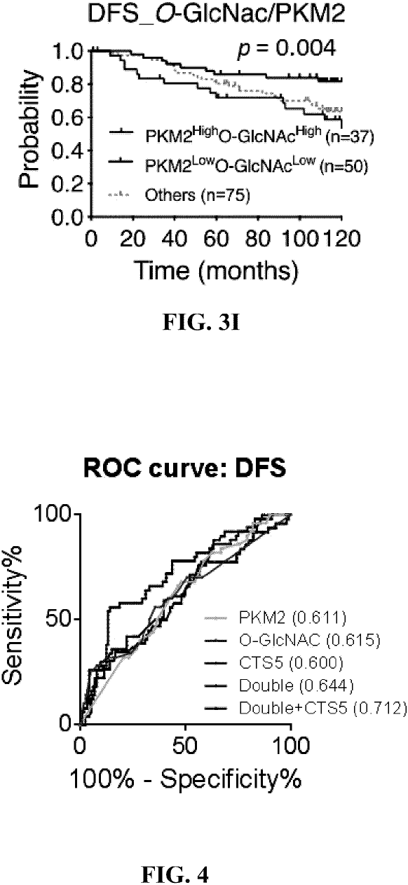

CTS5 which integrates 4 clinical properties (age, tumor size, grade, and node number) remains as a valuable system to predict late recurrences of breast cancer. Thus, we used logistic regression analysis to evaluate the risk of 10-year DFS based on the CTS5 score, as well as on the IHC status of PKM2 and O-GlcNAc. High expression of either O-GlcNAc (p = 0.006), or PKM2 (p = 0.026) was significantly associated with increased risk of disease progression, and a high score of CTS5 was also linked to an increased risk, yet with less significance (p = 0.073). Next, we built a model to combine the three variables. Overall, the expression of O-GlcNAc/PKM2 showed to provide extra information compared with the conventional CTS5 score in this model (see FIG. 4 and Table 2).

We then examined the ROC curves of the PKM2, O-GlcNAc, CTS5, PKM2-O-GlcNAc, and PKM2-O-GlcNAc-CTS5 models, respectively. FIG. 4 shows that the combined PKM2-O-GlcNAc-CTS5 model exhibited a greatly improved AUC value (AUC = 0.712) as compared with the CTS5 (AUC = 0.600) and PKM2-O-GlcNAc (AUC = 0.644) models. As shown in FIG. 4, individual factors are not accurate enough to evaluate the prognosis of breast cancer. Only when the three are combined, the AUC value (0.712) is significantly higher than other combinations, showing that the combination of the three may be used as a good indicator of the prognosis of breast cancer. Table 2 shows logistic regression analysis of O-GlcNAc, PKM2, and the CTS5 score based on 10-year DFS.

TABLE 2

| | coefficient | SE | p-value |
|---|---|---|---|
| O-GlcNAc | 0.007 | 0.003 | 0.018 |
| PKM2 | 0.005 | 0.002 | 0.028 |
| CTS5 Score | 0.525 | 0.215 | 0.015 |
| Constant | -3.883 | 1.002 | <0.001 |

p-value was calculated using the Wald test.

These results of this example suggest that the combined PKM2-O-GlcNAc-CTS5 model has superior discriminatory accuracy, offering an improved prognostic indicator for HR⁺/HER2⁻ luminal breast cancer.

In summary, the method and the kit of the present invention provide a new combination of clinical molecular characteristics for more effective evalution of the prognosis in a patient with breast cancer, especially for the luminal breast cancer that is HR positive and HER2 negative. In addition, the method of the present invention is more effective than using CTS5 alone, and can become a new metabolic prognostic biomarker, as well as a concomitant indicator of drug use.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for evaluating prognosis in a patient with breast cancer, comprising the following steps:
(a) detecting an expression level of pyruvate kinase in a sample from the patient;
(b) detecting an expression level of O-linked β-N-acetylglucosamine (O-GlcNAc) in the sample of step (a); and
(c) evaluating a clinical treatment score post-5 years (CTS5) of the patient; wherein high expression levels of both the pyruvate kinase and the O-GlcNAc, and high score of the CTS5 indicate that the patient has a poor prognosis.

2. The method according to claim 1, wherein the pyruvate kinase is pyruvate kinase isoenzyme M2 (PKM2).

3. The method according to claim 1, wherein the breast cancer is a luminal breast cancer that is hormone receptor (HR) positive and human epidermal growth factor receptor type 2 (HER2) negative.

4. The method according to claim 3, wherein the HR positive is selected from the group consisting of: estrogen receptor (ER) positive, progesterone receptor (PR) positive, and a combination thereof.

5. The method according to claim 1, wherein the expression level of pyruvate kinase in step (a) and the expression level of O-GlcNAc in step (b) are detected by an immunohistochemistry (IHC) method.

6. The method according to claim 1, wherein the prognosis is evaluated by disease-free survival (DFS).

7. The method according to claim 6, wherein the DFS is ten years.

8. The method according to claim 2, wherein the expression level of PKM2 and the expression level of O-GlcNAc are determined as high expression levels or low expression levels by analysis of Tumor Immune Estimation Resource (TIMER).

9. The method according to claim 8, wherein the expression level of PKM2 and the expression level of O-GlcNAc are determined as high expression levels based on top 25% of patients with breast cancer as criterion.

10. A kit for evaluating prognosis in a patient with breast cancer, comprising:
a first test reagent for detecting an expression level of pyruvate kinase in a sample from the patient;
a second test reagent for detecting an expression level of O-linked β-N-acetylglucosamine (O-GlcNAc), wherein the second test reagent interacts with the first test reagent; and
a third test reagent for evaluating a clinical treatment score post-5 years (CTS5), wherein the third test reagent interacts with the first test reagent and the second test reagent;
wherein high expression levels of both the pyruvate kinase and the O-GlcNAc, and high score of the CTS5 indicate that the patient has a poor prognosis.

11. The kit according to claim 10, wherein the pyruvate kinase is pyruvate kinase isoenzyme M2 (PKM2).

12. The kit according to claim 10, wherein the breast cancer is a luminal breast cancer that is hormone receptor (HR) positive and human epidermal growth factor receptor type 2 (HER2) negative.

13. The kit according to claim 12, wherein the HR positive is selected from the group consisting of: estrogen receptor (ER) positive, progesterone receptor (PR) positive, and a combination thereof.
